SEQUENCE LISTING (2 sequences)

SEQ ID NO: 1 - DNA, 349 bp, Hepatitis C virus
  misc_feature 299, 319: n is uncertain
ggatccggcgacactccaccatgaatcactcccctgtgaggaactactgtcttcacgcagaaagcgtctagccatggcgttagtatgagtgtcgtgcagcctccaggaccccccctcccgggagcgccatagtggtctgcggaaccggtgagtacaccggaattgccaggacgaccgggtcctttcttgataaacccgctcaatgcctggagatttgggcgtgcccccgcaagactgctagccgagtagtgttgggtcgcgaaaggccttgtggtactgcctgatagggtgcttgcgantgccccgggaggtctcgtanaccgtgcaccatgagcacgaatctggatcc

SEQ ID NO: 2 - protein, 112 aa, Hepatitis C virus
  MISC_FEATURE 95, 102: Xaa is uncertain
GSGDTPPITPLGTTVFTQKASSHGVSMSVVQPPGPPLPGAPWSAEPVSTPELPGRPGPFLDKPAQCLEIWACPRKTASRVVLGRERPCGTAGACXCPGRSRXPCTMSTNPGS

(19) United States
(12) Patent Application Publication
(10) Pub. No.: US 2006/0127923 A1
(43) Pub. Date: Jun. 15, 2006

(54) Internal ribosomal entry site mediated gene expression

(75) Inventors: Terrence Rabbitts, Cambridge (GB); Yoshihiro Yamada, Kyoto (JP); Grace Chung, Hong Kong (HK)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 11/191,806

(22) Filed: Jul. 28, 2005

Related U.S. Application Data
(63) Continuation of application No. PCT/GB04/00361, filed on Jan. 28, 2004.

(30) Foreign Application Priority Data
Jan. 29, 2003 (GB) 0302113.6

(51) Int. Cl. C12Q 1/68; G01N 33/574; C07H 21/04; C12P 21/06; C12N 9/64; C07K 14/82

(52) U.S. Cl. 435/6; 435/7.23; 435/69.1; 435/226; 435/320.1; 530/350; 536/23.5

(57) ABSTRACT

The present invention relates to an IRES element that is operably linked to one or more coding sequences, wherein the IRES element expresses said coding sequences in an endothelial cell.

This application is a Continuation of International Application No. PCT/GB04/000361, which was filed on 28 Jan., 2004, which designated the United States and was published in English, and which claims the benefit of United Kingdom Application GB0302113.6, filed 29 Jan. 2003. The entire teachings of the above applications are incorporated herein by reference.

### BACKGROUND

1. Field of Invention

The present invention relates to an internal ribosome entry site (IRES) element.

In particular, the present invention relates to an IRES element, for example, one or more IRES elements, operably linked to one or more coding sequences, wherein the IRES element expresses said coding sequence(s) in an endothelial cell.

The present invention further relates to vectors, methods, compositions and uses of the IRES element.

2. Background to the Invention

Specific expression of genes following delivery to target cells is a key objective for molecular based therapies, and blood vessel endothelial cells are important targets in a variety of clinical indications. The re-modelling of established vasculature (angiogenesis) occurs at specific times in normal conditions, such as wound healing or menstruation, and in a number of conditions, such as cancer, ischaemia, diabetic retinopathy and inflammatory diseases (1). In addition, neovascularisation, establishment of collateral circulation in ischaemic diseases and formation of granulation tissue in inflammatory diseases is a hallmark of these pathological conditions (1,2).

In cancer, angiogenesis is important to support in situ growth of primary solid tumours and also in metastasis where tumour cells traverse the infiltrating blood vessels (3), enter the blood stream and eventually forming metastatic deposits in remote locations. The process of angiogenesis has been advocated as an important point of therapeutic intervention in cancer (4) as depleting growing tumours of blood supply can inhibit their growth.

Many molecular targets will be confined to intracellular locations which require access of molecular therapeutics to the inside of the cell. This can be achieved with the use of viral vectors such as adenovirus (5) or lipid formulations which carry DNA vectors across the plasma membrane (6). These impose restrictions in that these are usually designed to express a single gene within the target cell. The use of internal ribosome entry sites (IRES) in bicistronic mRNAs has been a common strategy for dual gene expression but these structural elements in mRNA display developmental and cell type specificity (7,8).

It is often necessary to provide more than one gene, for example, a selectable marker, for the sustained expression of a therapeutic gene in cell. Hence, the ability to co-express multiple genes is an important consideration.

There are generally three ways in which two or more gene can be co-expressed: (1) separate promoters can be used to drive expression of different genes in the same vector. However, such constructs often suffer from the problem of promoter attenuation; (2) two different genes can be fused together in-frame to produce a chimeric protein, but this approach may not work for all proteins and can result in misfolding or mistargetting, for example; and (3) bicistronic constructs may be prepared in which two genes separated by an IRES element are expressed as a single transcriptional cassette under the control of a common upstream promoter. The intervening IRES sequence functions as a ribosome binding site for efficient cap-independent internal initiation of translation.

The incorporation of IRES elements into vectors represents a promising strategy to efficiently co-express several gene products, for example, in therapeutic settings.

However, the use of IRES elements for the co-expression of two or more genes also has problems. For example, the encephalomyocariditis virus (EMCV) IRES element results in less efficient expression of the downstream gene compared with the cap-dependent translation of the upstream gene (Hum Gene Ther (1995) 6, 905-915; Hum Gene Ther (1998) 9, 287-293). In addition, most IRES elements are generally tissue-specific and most work poorly in endothelial cells. This may be due to the absence of protein factors in these cells, which bind the IRES element.

Thus, there is a need in the art for IRES elements that efficiently express coding sequences in endothelial cells.

### SUMMARY OF THE INVENTION

The present invention is based in part upon the finding that expression of a coding sequence in an endothelial cell can be mediated using an IRES element, for example an HC-IRES element. Surprisingly, the expression of a coding sequence in an endothelial cell can be mediated using an HC-IRES element, but not other viral IRES elements—such as EMC-IRES.

Therefore, the HC-IRES, but not EMC-IRES, can be used for protein synthesis in endothelial cells, providing the means to co-express proteins in blood vessel endothelial of clinical conditions which depend on angiogenesis.

According to a first aspect, the present invention relates to a vector comprising an endothelial cell ligand and one or more IRES elements operably linked to one or more coding sequences, wherein the IRES element expresses said coding sequences in an endothelial cell.

The incorporation of one or more IRES elements operably linked to one or more coding sequences into a vector allows for the expression of coding sequences in endothelial cells. Moreover, the inclusion of an endothelial cell ligand allows for the specific expression of one or more coding sequences in endothelial cells.

Advantageously, the IRES element of the present invention, when operably linked to one or more coding sequences, for example, one or more therapeutic genes, provides the means to express one or more coding sequences under clinical conditions which involve endothelial cells. For example, the invention allows coding sequences to be expressed in angiogenic conditions in nascent blood vessels.

According to a second aspect, the present invention provides a method for expressing one or more coding sequences comprising the steps of: (a) identifying an IRES element that expresses one or more coding sequences in an endothelial cell; (b) inserting the IRES element into a vector; (c) transfecting the vector in to an endothelial cell; and (d) providing for expression of the one or more coding sequences in the endothelial cell.

In a third aspect, the present invention relates to a method for preparing a vector for the expression of one or more coding sequences in an endothelial cell comprising the step of operably linking an IRES element to one or more coding sequences in a vector.

The method according to the third aspect of the present invention may additionally comprise the steps of: transfecting the vector into an endothelial cell; providing for expression of the one or more coding sequences; and determining whether the coding sequences are expressed in the endothelial cell.

In a fourth aspect, the present invention relates to a method for identifying an IRES element that expresses of one or more coding sequences in an endothelial cell comprising the steps of: (a) operably linking an IRES element to one or more coding sequences in a vector; (b) transfecting the vector into an endothelial cell; (c) providing for expression of the one or more coding sequences; and (d) determining whether the coding sequences are expressed in the endothelial cell.

This method can be used to identify other IRES elements, for example, IRES elements of viral or cellular origin, that express one or more coding sequences in an endothelial cell.

In a fifth aspect, the present invention relates to a method for delivering one or more coding sequences to an endothelial cell, which comprises the step of transducing the endothelial cell with a vector according to the present invention.

In a sixth aspect, the present invention relates to the use of an IRES element in the expression of one or more coding sequences in an endothelial cell.

In a seventh aspect, the present invention relates to a method for treating or preventing a disease in a subject, which comprises the step of administering a vector according to the present invention to a subject.

In an eighth aspect, the present invention relates to a pharmaceutical composition comprising a therapeutically effective amount of a vector according to the present invention, and optionally a pharmaceutically acceptable carrier, diluent, excipient or adjuvant or any combination thereof.

In a ninth aspect, the present invention relates to a vector according to the present invention for use in the treatment of a disease.

In a tenth aspect, the present invention relates to the use of a vector according to the present invention, in the manufacture of a pharmaceutical composition for the treatment of a disease.

In accordance with the above-mentioned aspects, preferably, the IRES element comprises SEQ ID No. 1 or SEQ ID No. 2.

Preferably, the IRES element is a HC-IRES element.

Preferably, one or more IRES elements are operably linked to two or more coding sequences. This allows for the expression of multiple coding sequences in endothelial cells, which may be particularly advantageous for the treatment of diseases.

Preferably, the coding sequence(s) are therapeutic genes.

Preferably, the coding sequence(s) are expressed in endothelial cells in vitro or in vivo.

Preferably, the endothelial cells are diseased.

Preferably, the disease is an angiogenesis-dependent disease. More preferably, the angiogenesis-dependent disease is caused by excessive angiogenesis or insufficient angiogenesis. Most preferably, the disease is selected from: cancer, ischaemic, diabetic retinopathy, an inflammatory disease, age-related macular degeneration, rheumatoid arthritis, psoriasis, coronary artery disease, stroke, and delayed wound healing.

Preferably, the endothelial cell ligand is a tumour endothelial cell ligand.

Preferably, the endothelial cells are human endothelial cells.

Preferably, at least one of the coding sequences is under the control of an upstream promoter.

Preferably, the vector is a viral vector.

### DESCRIPTION OF THE FIGURES

FIG. 1

Activity of IRES elements in mouse embryo vascular endothelium. (A) Constructs for homologous recombination. Top line: The partial restriction map of the mouse Lmo2 gene shows the location of Lmo2 exons 2 and 3, together with the two probes (A and B) used to detect homologous recombination (10). Middle line shows a map of the targeting vector pKO5tk (10) which has a BamHI restriction site introduced within exon 2 to facilitate cloning of exogenous elements into Lmo2. Bottom line shows the maps of the lacZ gene insertions cloned in the exon2 BamHI site for Lmo2-lacZ (in-frame lacZ fusion with the 5′ end of Lmo2) (20), HC-IRES and EMC-IRES. (B) Whole mount X-gal staining of mouse embryos at embryonic stages E9.5, E10.5 and E12.5 showing expression of β-galactosidase from the Lmo2 gene in de novo capillary formation (vasculogenesis) and endothelial re-modelling (angiogenesis) during mouse embryo development. Wt=wild type C57B16

FIG. 2

Histology of Lmo2 lacZ knock-in E10.5 embryos shows co-expression of β-galactosidase and the pan-endothelial marker CD31. E10.5 embryo specimens were whole mount stained with X-gal (FIG. 2), sectioned (4 μM), counter stained with haematoxylin and eosin. CD31 protein expression was detected in serial sections using anti-CD31 antibody and peroxidase. The montage shows embryo sections from each indicated Lmo2 knock-in mouse line, or wild type (wt) controls stained only with Xgal (left) or co-stained with X-gal and anti-CD31 (right). Arrowheads indicate endothelial cells lining blood vessel walls.

FIG. 3

Expression of β-galactosidase from hepatitis C IRES in Lewis lung solid tumours. Lewis lung carcinoma cells were implanted sub-cutaneously in the Lmo2 HC-IRES knock-in mouse line and Lmo2-lacZ or wild type (wt) controls. (A) The vasculature of the tumours growing in the recipient mice comes from the latter and therefore the endothelial cells will be expressing the Lmo2-reporter of the recipient. In the case of Lmo2-lacZ and HC-IRES mouse lines, the expression of β-galactosidase is detected using Xgal substrate. (B) After tumour growth, solid tumours were whole mount stained with X-gal and 4 μM sections made for examination of tumour vascular endothelium which forms by sprouting of existing endothelium from recipient mice. Arrowheads indicate endothelial cells lining blood vessel walls.

### DETAILED DESCRIPTION OF THE INVENTION

IRES Element

Most eukaryotic mRNAs are translated primarily by ribosome scanning. First, the 40S ribosomal subunit with its associated initiation factors binds to the 5′7-methylguanosine-cap structure of the mRNA to be translated. The complex then scans in the 3′ direction until an initiation codon in a favourable context is encountered, at which point protein translation is initiated. According to this model, the presence of a 5′ untranslated region (UTR) with strong secondary structure and numerous initiation codons would present a significant obstacle, leading to inefficient translation by ribosome scanning. Ribosome reinitiation, shunting, and internal ribosome binding are secondary mechanisms of translation initiation that alleviate the requirement for ribosome scanning and allow translation to proceed in a cap-independent manner.

IRES elements have developed to allow viruses to express more than one gene per mRNA. The cell types in which this activity occurs are variable and depend on the virus. IRES elements bind to cellular protein factors and these can be cell-type specific lending an internal degree of specificity to the system.

IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES elements is presented by Mountford and Smith (TIG May 1995 vol 1, No 5:179-184).

According to WO-A-97/14809, IRES elements are typically found in the 5′ non-coding region of genes. In addition to those in the literature they can be found empirically by looking for genetic sequences that affect expression and then determining whether that sequence affects the DNA (i.e. acts as a promoter or enhancer) or only the RNA (acts as an IRES element).

The term “IRES element” includes any sequence or combination of sequences, which work as or improve the function of an IRES element.

A number of different IRES elements are known including those from encephalomyocarditis virus (EMCV) (Ghattas, I. R., et al., Mol. Cell. Biol., 11:5848-5859 (1991); polio virus (PV) (Pelletier and Sonenberg, Nature 334: 320-325 (1988)); and hepatitis C virus (see Gallego and Varani (2002) Biochem. Soc. Transac. 30 p140-145; BiP protein (Macejak and Samow, Nature 353:91 (1991)); the Antennapedia gene of drosphilia (exons d and e) (Oh, et al., Genes & Development, 6:1643-1653 (1992)); eukaryotic initiation factor 4G (EIF4G) (J. Biol. Chem.(1998) 273, 5006-5012); and vascular endotheial growth factor (VEGF) (Mol Cell Biol (1998) 18, 3112-3119). One skilled in the art will appreciate that this list is not intended to be exhaustive.

In accordance with the present invention, one or more IRES elements are operably linked to one or more coding sequences.

When used in this configuration, the IRES element may be used for homologous recombination to integrate one or more IRES elements operably linked to one or more nucleic acid sequences into a chromosome—such as a chromosomal locus. This may be achieved by using, for example, polycistronic-targeting vectors incorporating both IRES-coding sequence cassettes and IRES-selectable marker elements. In the case of non-expressed genes, the selectable marker may be promoter driven. The selectable marker cassette may subsequently be excised by site-specific recombination as described by Jung et al. (1993) Science 259, 984-897.

Sequential targeting (Jung et al. (1993) Science 259, 984-897) may also be used whereby a counter-selectable marker is incorporated in an initial homologous recombination event, followed by substitution with the IRES-coding sequence in a second step. Accordingly, a nucleotide sequence, for example, a coding sequence, may be placed under the full regulatory control of an endogenous genomic locus.

In a preferred embodiment of the present invention, one or more IRES elements are operably linked to two or more coding sequences.

In order for the IRES element to be capable of initiating translation of a coding sequence, the IRES element should be located between coding sequences, which are under the control of a common upstream promoter. The methionine start codon of the IRES should be in frame with the input coding region. Coupled transcription of both coding sequences occurs, followed by cap-independent initiation of translation of the first coding sequence and IRES-directed cap-independent translation of the second coding sequence. In other words there will always be one fewer IRES elements than the coding sequences. For example, for bi- and tri-cistronic sequences, the order may be as follows:
Promoter-coding sequence ₁-IRES₁-coding sequence₂
Promoter-coding sequence ₁-IRES₁-coding sequence₂-IRES₂-coding sequence 3

The use of an IRES element in therapeutic settings is desirable where delivery of one or more distinct proteins is required, such as in the angiogenesis target of cancer therapy.

The IRES elements may be of viral origin or cellular origin.

Preferably, the IRES element is of viral origin. More preferably, the IRES element is from the family Flaviviridae. More preferably, the IRES element is from the genera Hepacivirus. Most preferably, the IRES element is from the hepatitis C virus (HC-IRES).

HC-IRES Element

Preferably, the IRES element comprises SEQ ID No. 1. More preferably, the IRES element consists of SEQ ID No. 1.

Preferably, the IRES element comprises SEQ ID No. 2. More preferably, the IRES element consists of SEQ ID No. 2.

These sequences encode the hepatitis C virus IRES (HC-IRES) element.

Hepatitis C virus (HCV) contains an IRES element located in the 5′ untranslated region of the genomic RNA that drives cap-independent initiation of translation of the viral message. The approximate secondary structure and minimum functional length of the HCV IRES element is known, and extensive mutagenesis has established that nearly all secondary structural domains are critical for activity.

HCV-IRES element medicated translation initiation only requires interaction between the IRES element and two components of the 43S particle, the 40S subunit and eIF3. This interaction results in the direct recognition of the viral start codon and the initiation of protein synthesis.

The IRES element encompasses most of the 5′UTR of the HCV RNA and is highly conserved compared with the rest of the viral genome, suggesting that it plays an essential role in the viral life cycle. The HCV IRES element contains four conserved secondary structure domains. Electron microscopy studies have advanced the understanding of the overall structural organisation of the HCV IRES element (Spahn et al. (2001) Science 291, 1959-1962; Beales et al. (2001) RNA 7, 661-670).

The HCV IRES element has been described and reviewed in, for example, Biochem. Soc. Transac. (2002) 30 pl40-145; J Viral Hepat 1999 6(2), 79-87; Princess Takamatsu Symp 1995;25:99-1 10; and J Virol 1992 Mar;66(3):1476-83.

Nucleotide Sequence

The present invention involves the use of nucleotide sequences, which may be available in databases. These nucleotide sequences may be used to express amino acid sequences.

Thus, the nucleotide sequence can be, for example, a synthetic RNA/DNA sequence, a recombinant RNA/DNA sequence (i.e. prepared by use of recombinant DNA techniques), a cDNA sequence or a partial genomic DNA sequence, including combinations thereof.

The nucleotide sequence may be double-stranded or single-stranded. In addition, the RNA/DNA sequence may be in a sense orientation or in an anti-sense orientation. Preferably, it is in a sense orientation.

Preferably, the nucleotide sequence(s) are coding sequences ie. the portion of the nucleotide sequence that is translated into protein.

Preferably, the coding sequence comprises a therapeutic gene.

As used herein, the term “therapeutic gene” refers to any gene that can be used for the modulation and/or treatment and/or prevention of a disease—such as a disease of an endothelial cell.

Suitable therapeutic genes may include, but are not limited to: sequences encoding enzymes, cytokines, chemokines, hormones, antibodies, anti-oxidant molecules, engineered immunoglobulin-like molecules, a single chain antibody, fusion proteins, immune co-stimulatory molecules, immunomodulatory molecules, anti-sense RNA, a toxin, a conditional toxin, an antigen, tumour suppresser proteins and growth factors, membrane proteins, vasoactive proteins and peptides, anti-viral proteins and ribozymes, and derivatives thereof (such as with an associated reporter group). The coding sequence may also encode pro-drug activating enzymes.

Suitable therapeutic genes may also include angiogenic growth factors—such as, but not limited to, Angiogenin, Angiopoietin-1, Del-1, Fibroblast growth factors: acidic (aFGF) and basic (bFGF), Follistatin, Granulocyte colony-stimulating factor (G-CSF), Hepatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN), Proliferin, Transforming growth factor-alpha (TGF-alpha), Transforming growth factor-beta (TGF-beta), Tumor necrosis factor-alpha (TNF-alpha), and/or Vascular endothelial growth factor (VEGF)/vascular permeability factor (VPF)

Suitable therapeutic genes may also include angiogenesis inhibitors—such as, but not limited to, Angiostatin (plasminogen fragment), Antiangiogenic antithrombin III, Cartilage-derived inhibitor (CDI), CD59 complement fragment, Endostatin (collagen XVIII fragment), Fibronectin fragment, Gro-beta, Heparinases, Heparin hexasaccharide fragment, Human chorionic gonadotropin (hCG), Interferon alpha/beta/gamma, Interferon inducible protein (IP-10), Interleukin-12, Kringle 5 (plasminogen fragment), Metalloproteinase inhibitors (TIMPs), 2-Methoxyestradiol, Placental ribonuclease inhibitor, Plasminogen activator inhibitor, Platelet factor4 (PF4), Prolactin 16kD fragment, Proliferin-related protein (PRP), Retinoids, Tetrahydrocortisol-S, Thrombospondin-1(TSP-1), Transforming growth factor-beta (TGF-b), Vasculostatin and/or Vasostatin (calreticulin fragment).

The use of at least one IRES element, for example, 2, 3, 4, 5, 6, 7, 8, 9 or even 10 or more IRES elements, in accordance with the present invention means that one or more coding sequences—such as 2, 3, 4, 5, 6, 7, 8, 9 or even 10 or more coding sequences—may be expressed in an endothelial cell.

The coding sequence may encode all or part of a protein, or a mutant, homologue or variant thereof. For example, the coding sequence may encode a fragment of a protein which is capable of functioning in vivo in an analogous manner to the wild-type protein.

Two different coding sequences that are operably linked to a regulatory sequence may be fused together in-frame to express a chimeric protein.

Operably Linked

As used herein, the term “operably linked” means a regulatory sequence, for example, an IRES element or a promoter, and one or more coding sequences that are in a relationship permitting them to function in a manner that results in the expression of the coding sequence.

The regulatory sequence “operably linked” to one or more coding sequences is ligated in such a way that expression of the coding sequence is achieved under conditions compatible with the regulatory sequences.

Typically, the regulatory sequences will be ligated in frame with the coding sequence.

Endothelial Cell

As used herein, the term “endothelial cell” refers to cells lining blood vessels and lymphatics.

Endothelial cells are typically located at the interface between the blood and the vessel wall. The cells are in close contact and form a slick layer that prevents blood cell interaction with the vessel wall as blood moves through the vessel lumen.

Endothelial cells may perform the following functions: they may act as a selective barrier to the passage of molecules and cells between the blood and the surrounding bodily tissue eg. the blood-brain barrier and the barrier between the central nervous system and the rest of the body; they may play an essential role in summoning and capturing leukocytes to the site of an infection; they may play an important role in the mechanics of blood flow; and they may regulate coagulation of the blood at the site of a trauma.

The endothelial cells may be provided in vitro—such as an endothelial cell line. Examples of such cell lines include, but are not limited to, human unbiblical cord endothelial cells and Bend3 cells (a rat brain endothelial cell line).

Preferably, the endothelial cells are provided in vivo.

Preferably, the endothelial cells are human cells.

Blood vessel endothelial cells may be cells of variable levels of differentiation, for example, CD31+. ES cell culture differentiation may be achieved with FLK1+CD31-cells and so in vivo cells of this phenotype are also endothelial.

Endothelial Cell Ligand

As used herein, the term “endothelial cell ligand” refers to one or more moieties that can bind to a target site—such as a surface marker or a receptor—expressed by an endothelial cell.

Preferably, the target site is expressed by a diseased endothelial cell.

Targeting of vectors specifically to endothelial cells—such as diseased endothelial cells—may be mediated by means of one or more endothelial cell ligands incorporated in to a vector by genetic, chemical, biological or immunological methods. The success of the specific targeting depends on a number of factors—such as the specificity of the endothelial cell ligand for the endothelial cell to be targeted and the degree of affinity of the binding reaction.

The endothelial cell ligand may be a single entity or it may be a combination of entities. The endothelial cell ligand may be an organic compound or other chemical. The endothelial cell ligand may be a compound, which is obtainable from or produced by any suitable source, whether natural or artificial. The endothelial cell ligand may be an amino acid molecule, a polypeptide, a peptide or a chemical derivative thereof, or a combination thereof. The endothelial cell ligand may even be a polynucleotide molecule—which may be a sense or an anti-sense molecule. The endothelial cell ligand may even be an antibody.

If the endothelial cell ligand is an antibody then it may be a complete antibody, an antibody fragment, or an antibody peptide. Thus, by way of example, an endothelial cell ligand may include Fv, ScFv, Fab′ and F(ab′)₂, monoclonal and polyclonal antibodies, engineered antibodies including chimeric, CDR-grafted and humanised antibodies, artificially selected antibodies produced using phage display or alternative techniques.

Preferably, cells other than diseased endothelial cells should not expose or present the structure to which the endothelial cell ligand binds or should expose or present only a very low number of those structures per cell.

Preferably, the number of structures per diseased endothelial cell should be high enough to allow binding with high avidity of sufficient amounts of endothelial cell ligand.

Assuming a transfection efficiency (vector target cell ratio) of 10:1, preferably, the number of endothelial cell ligands specific target sites on the cell membrane should be higher than 10/cell. To increase the avidity of the binding reaction, the vector particle should carry as much endothelial cell ligand as possible and the target cell should express an excess of membrane receptor structures. Preferably, the number of receptors per cell is 10²/cell or more.

By way of example, in the case of tumour endothelial cells, endothelial cells outside of the tumour tissue or blood cells or other parenchymal cells protruding into the blood stream should either not expose the cell membrane structure to which the endothelial cell ligand binds or should expose only a very low number of those structures per cell.

Natural ligands may competitively inhibit binding of the endothelial cell ligand to its target site. Preferably, the endothelial cell ligand that is selected has either significantly higher affinity for binding to its target site than any natural ligand or the competing natural ligand is present in blood in trace amounts only.

Preferably, the vector according to the present invention that comprises the endothelial cell ligand is intemalised in the diseased endothelial cell.

Many surface markers to which the endothelial cell ligand may bind have been reported. The enhanced expression of many of those endothelial cell markers requires activation of endothelial cells by mediators such as IL-1 or TNFα. As a consequence, many surface markers expressed by activated endothelial cells are more strongly expressed in diseased endothelial cells, than by normal (non-diseased) cells.

The surface markers may not be exclusively expressed by diseased endothelial cells but may also be expressed by macrophages, dendritic cells, lymphocytes, tissues cells or different organs or tumour cells. However, the significant differences in the level of cell surface marker expression between normal endothelial cells and diseased endothelial cells—such as tumour endothelial cells—and the direct accessibility to intravascularly applied vectors to endothelial cells lining tumours provides for the use of these markers for specific targeting of diseased endothelial cells.

Many different surface markers expressed on activated endothelial cells have been reported and include, but are not limited to, receptors for growth factors: TIE-2, VEGF R-I, -II, -III, PDECGF-R, FGF-R-I, -II, -III, -IV, EGF-R, PDGF-R, TGFβ-R-I, -II, HGF-R; receptors for cytokines/chemokines: IL-I-R-I, -II, IL-3-R, IL-4-R, IL-6-R, IL-8-R-I, -II, IL-12-R, LIF-R, TNF-R, IFNγ-R, IFNα, β-R, G-CSF-R, M-CSF-R, GM-CSF-R, Oncostatin-M-R; receptors for blood plasma components: CNTF, Fcγ-RII, LPS-R, OxyLDL-R, Tsp-1-R, pgp IV, uPA-R, thrombomodulin, angiostatin rec., factor VIII related antigen; cell adhesion molecules: PECAM-1, ICAM-1, ICAM-2, ICAM-3, β3 integrin, H-CAM, sLex, VCAM-1, GMP-140, ELAM-1, MCP, cell CAM-105, VLA-1, -2, 5; and other cell membrane proteins: TF and Thy-1.

Many different surface markers expressed on diseased tumour endothelial cells have also been reported and include, but are not limited to, TAL-1 (J. Pathol. (1996) 178, 311-5), VEGF/VEGR R-II complex (Cancer Res. (1998) 58, 1952-9), VEGR-I (Am J. Pathol. (1998) 153, 1239-48), VEGR-II (Mol. Endocrinol (1995) 9, 176-70), Tie-2 (PNAS (1998) 95, 8829-34), Endoglin (J. Immunol. (1996) 156, 565-73), α3/β3 integrin (Int J Cancer (1997) 71, 320-4), angiostatin receptor (PNAS (1999) 96, 2811-6), E-selectin/ELAM-1 (Gene Ther. (1999) 6, 801-7), PSMA (Cancer Res (1999) 148, 465-72), CD44 (Blood (1997) 90, 1150-9), ICAM-3 (Am J Pathol (1996) 148, 465-72), CD40 (Cancer Res. (1997) 57, 891-9), and TF (PNAS USA (1999) 96, 8161-6).

Disease

In a preferred embodiment of the present invention, the endothelial cells are diseased.

As used herein, the term “disease” refers to any anatomical abnormality or impairment of the normal functioning of an endothelial cell.

The disease may be caused by environmental factors—such as malnutrition or toxic agents, infective agents—such as bacteria or viruses, genetic disease, or any combination of these factors.

Preferably, the disease is an angiogenesis-dependent disease.

In many serious disease states, the body loses control over angiogenesis. Angiogenesis-dependent diseases may result when new blood vessels either grow excessively or insufficiently.

There are number of important clinical indications where angiogenesis is an important consequence. For example, neovascularisation occurs around malignant tumours in order to supply enough oxygen for rapidly dividing cells. In chronic inflammatory diseases—such as rheumatoid arthritis—sustained inflammation results in the formation of vascular rich granulation tissues in the synovial membrane. Thus in these circumstances, preventing blood vessel remodelling and neovascularisation is a potential therapeutic approach.

In solid tumour therapy, internal angiogenesis protein targets—such as LMO2—may be accessible by introduction of vectors expressing targeted dual blocking reagents aimed at prohibiting function of the target protein in distinct ways (e.g. using an intracellular antibody fragment and a peptide aptamer). Methods for delivery of vectors to specific cells in vivo are becoming more effective and specific ways of delivering vectors into endothelial cells have been reported (Sedlacek (2001), Critical Reviews in Oncology/Hematology 37 169-215). Combining these delivery methods with the ability to efficiently express one or more proteins which can combat the function of specific targets is a very promising approach to anti-angiogenesis therapies.

Excessive angiogenesis may occur in diseases such as cancer, diabetic blindness, age-related macular degeneration, rheumatoid arthritis, and psoriasis. In these conditions, new blood vessels feed diseased tissues, destroy normal tissues, and in the case of cancer, the new vessels allow tumor cells to escape into the circulation and lodge in other organs (tumor metastases). Excessive angiogenesis may also occur when diseased cells produce abnormal amounts of angiogenic growth factors, overwhelming the effects of natural angiogenesis inhibitors.

Accordingly, the use of an IRES element in the expression of one or more coding sequences in an endothelial cell may be used for the expression of coding sequences that modulate excessive angiogenesis.

Insufficient angiogenesis may occur in diseases such as coronary artery disease, stroke, and delayed wound healing. In these conditions, inadequate blood vessels grow, and circulation is not properly restored, leading to the risk of tissue death. Insufficient angiogenesis occurs when the tissue cannot produce adequate amounts of angiogenic growth factors.

Accordingly, the use of an IRES element in the expression of one or more coding sequences in an endothelial cell may be used to stimulate new blood vessel growth with growth factors.

More preferably, the disease is selected. from: cancer, ischaemic, diabetic retinopathy, an inflammatory disease, age-related macular degeneration, rheumatoid arthritis, psoriasis, coronary artery disease, stroke, and delayed wound healing.

Vectors

The IRES element operably linked to one or more coding sequences may be prepared and/or delivered to a target site—such as a diseased endothelial cell—using a genetic vector.

As it is well known in the art, a vector is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising nucleotide sequences and/or expressing the proteins encoded by the nucleotide sequences. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitro expression.

The term “transformation vector” means a construct capable of being transferred from one species to another.

The vectors of the present invention may be transformed or transfected into a suitable host cell, for example, an endothelial cell, to provide for expression of one or more nucleotide sequence(s). This process may comprise culturing a host cell transformed with a vector under conditions to provide for expression by the vector of a nucleotide sequence encoding the protein, and optionally recovering the expressed protein. In some instances, the expression of a nucleotide sequence encoding the protein may be under the control of an inducible promoter, such that expression must be induced with, for example, IPTG.

The vectors may be for example, plasmid or virus vectors.

In a preferred embodiment, the vector is a viral vector.

In recent years, viruses, for example, retroviruses have been proposed for use in gene therapy.

Gene therapy includes any one or more of: the addition, the replacement, the deletion, the supplementation, the manipulation etc. of one or more nucleotide sequences in. General teachings on gene therapy are available in the art.

The vectors may comprise an endothelial cell ligand, as described herein.

Typically, the vectors will comprise one or more origins of replication—such as pUC ori, SV40 ori, and fl ori.

Typically, the vectors will contain one or more selectable marker genes, for example an ampicillin resistance gene in the case of a bacterial plasmid or a neomycin resistance gene for a mammalian vector.

Optionally, the vectors may comprise one or more promoters for the expression of one or more coding sequences—such as a selectable marker or a reporter—and optionally regulators of the promoters.

In a vector comprising a promoter operably linked to first coding sequence and an IRES element operably linked to a second coding sequence, the promoter will be located upstream of the IRES element, as described herein. Typically, in this type of vector two or more coding sequences will be expressed.

Two or more IRES elements may be included in the same vector, for the expression of two or more coding sequences.

Two different coding sequences may even be fused together in flame to produce a chimeric protein resulting in the expression of both coding sequences simultaneously.

Cloning vectors and transgene expression vectors comprising a promoter operably linked to first coding sequence and an IRES element operably linked to a second coding sequence according to the present invention may also be used as: (1) vectors for functional screening of cDNA libraries; (2) co-expression of fusion partners in a two-hybrid cloning system; and (3) for co-expression of a reporter and selectable marker fusion gene (TIG (1995) 11, 179-184).

The use of a IRES-based vector that does not comprise a promoter may provide for a major enrichment for homologous recombination events in gene targeting (Genes Dev (1988) 2, 1353-1363). Typically, each IRES will only support the protein synthesis of one coding region. Therefore, for the expression of more than one coding sequence, more than one IRES element will be needed. For example, for the expression of two coding sequences, two IRES elements will typically be required.

The exploitation of one or more IRES sequences operably linked to one or more coding sequences may improve this situation by, for example, simplifying the design of targeting vectors. By way of example, exploitation of IRES-linked selectable markers may allow subtle structural or regulatory alterations to be introduced into specific genes. Such alterations may include, but are not limited to, gene deletion or disruption, introduction of mutations and upregulation of gene expression (TIG (1995) 11 p179-184).

By way of example, a vector for homologous recombination may be constructed as follows. The plasmid may be based on pKO5tk, which has a unique BamHI restriction site mutated into exon 2. The vector is prepared by inserting the HC-IRES-LacZ-MC1neopA cassette into the BamHI site of the pKO5tk. A 400bp BamHI fragment including the IRES element from the hepatitis C virus vector pRT8 is first cloned into the BglII-BamHI sites of a modified pBSpt vector (pBspt-BGB4) to generate the precursor pBSpt-HC-IRES element with a unique BamHI site into which is cloned the lacZ gene and pMC1-neo-pA.

Promoter

In accordance with the present invention, one or more coding sequences are operably linked to a promoter, which is capable of providing for the expression of a coding sequence, such as by a chosen host cell.

The term “promoter” is used in the normal sense of the art, e.g. an RNA polymerase binding site.

Suitable promoting sequences may be derived from various sources, including, but not limited to bacteria, fungi and yeast. Preferably, suitable promoting sequences are strong promoters derived from the genomes of viruses—such as polyoma virus, adenovirus, fowlpox virus, bovine papilloma virus, avian sarcoma virus, cytomegalovirus (CMV), retrovirus and Simian Virus 40 (SV40)—or from heterologous mammalian promoters—such as the actin promoter or ribosomal protein promoter.

Preferably, the promoter is a cytomegalovirus (CMV) promoter.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the first nucleotide sequence. For example, suitable other sequences include the Sh1-intron or an ADH intron. Other sequences include inducible elements—such as temperature, chemical, light or stress inducible elements. Transcription of a coding sequence may also be increased further by inserting an enhancer sequence into the vector. Enhancers are relatively orientation and position independent, however, one will typically employ an enhancer from a eukaryotic cell virus—such as the SV40 enhancer on the late side of the replication origin (bp 100-270) and the CMV early promoter enhancer. The enhancer may be spliced into the vector at a position 5′ or 3′ to the promoter, but is preferably located at a site 5′ from the promoter.

Identifying an IRES Element

In a further aspect, the present invention relates to a method for identifying an IRES element that expresses of one or more coding sequences in an endothelial cell.

An IRES element to be tested for its ability to express one or more coding sequences in an endothelial cell may be operably linked to any coding sequence(s).

Preferably, the coding sequence(s) express one or more proteins that can be detected. Such proteins may be known as reporters. By way of example, the coding sequence may express β-galactosidase, or may express a fluorescent protein—such as red fluorescent protein or cyan fluorescent protein.

Preferably, the vector comprises an origin of replication for replication in mammalian cells, and/or bacterial cells and a selection marker—such as the antibiotic—resistance cassette and so the plasmid can be selected.

Advantageously, the vector may comprise one or more promoters—such as a CMV promoter. In order for the IRES element to be capable of initiating translation of a coding sequence, the IRES element should be located between coding sequences, which are under the control of a common upstream promoter. This enables coupled transcription of both genes, followed by cap-independent initiation of translation of the first coding sequence and IRES-directed cap-independent translation of the second first coding sequence.

Preferably, the one or more coding sequences expressed by the promoter(s) can be detected. More preferably, the coding sequence(s) expressed by the promoter(s) express one or more proteins that can be detected and are different to the coding sequence(s) that are expressed from the one or more IRES elements.

Advantageously, the use of one or more promoters operably linked to one or more coding sequences in the vector may provide a control to compare the levels of expression of the coding sequences from the promoter versus the levels of expression of the coding sequences from the IRES element under test.

The vector may even comprise a plurality of IRES elements operably linked to a plurality of coding sequences. Preferably, at least one of the IRES elements is the HC-IRES element fused to a coding sequence that expresses one or more proteins that can be detected.

Preferably, the protein that is expressed from the coding sequence fused to the HC-IRES element, is different to any of the other proteins that are expressed and so the expression of the protein can be attributed to the HC-IRES element. In this manner, the HC-IRES element provides a suitable control to identify IRES elements that express a coding sequence more or less efficiently than the HC-IRES element of the present invention.

The vector is transfected into an endothelial cell using various methods known in the art, as described herein. By way of example, cells may be transfected using liposome delivery of the expression vectors using lipofectamine 2000.

The level of expression of the coding sequences in an endothelial cell may be determined using various methods known in the art. The exact method used will depend upon the type of detectable protein that is expressed. For example, if the protein that is expressed is β galactosidase, then cells may be whole mount stained with X-gal.

In addition to determining the level of expression of the protein fused to the IRES element in an endothelial cell in vitro, the level of expression may also be determined in vivo.

By way of example, plasmids may be constructed for homologous recombination in a gene expressed in endothelial cells, such as, but not limited to Lmo2. A knock-in targeting clone may be prepared by inserting an HC-IRES-LacZ-MC1neopA cassette into the BamHI site of a suitable vector—such as pKO5tk. Cells may be transfected with the vector, targeted clones characterised by Southern filter hybridisation and then injected into blastocysts. Chimaeric mice may then be generated, from which germ-line transmission is obtained by breeding male chimaeras with females. Timed matings may be set up between heterozygous mice and wild type mice. At the appropriate times, the pregnant females are euthanased, embryos removed and expression is determined, for example, using whole mounts stained with X-gal to detect β-galactosidase. Post-fixed embryos may be sectioned after wax embedding.

Sections are then mounted on microscope slides and counter stained. Detection of endothelial cells may be achieved using various endothelial cell markers - such as PECAM (CD31) which is performed using MEC13.3 anti-CD31 antibody (Pharmingen) by the Avidin-Biotin conjugated peroxidase method.

Reporters

A wide variety of reporters may be used in accordance with the present invention with preferred reporters providing conveniently detectable signals (e.g. by spectroscopy). By way of example, a reporter gene may encode an enzyme which catalyses a reaction, which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts, which are then identified when bound to oligonucleotide probes.

For example, the production of the reporter molecule may be measured by the enzymatic activity of the reporter gene product, such as β-galactosidase.

A variety of protocols are available—such as by using either polyclonal or monoclonal antibodies specific for a protein to be detected. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on polypeptides is preferred, but a competitive binding assay may be employed. These and other assays are described, among other places, in Hampton R et al (1990, Serological Methods, A Laboratory Manual, APS Press, St Paul Minn.) and Maddox DE et al. (1983) J Exp. Med. 15 8:121 1).

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. Also, recombinant immunoglobulins may be produced as shown in U.S. No. 4,816,567.

Additional methods to quantify the expression of a particular molecule include radiolabeling (Melby PC et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Delivery

Aspects of the present invention relate to the delivery of a vector comprising an IRES element for use in the expression of one or more coding sequences in an endothelial cell in vivo.

The vector may be delivered alone or in combination with one or more further entities.

As used herein the term “delivery” includes delivery by viral or non-viral techniques.

Non-Viral Delivery

Non-viral delivery mechanisms include, but are not limited to transfection, lipid mediated transfection, liposomes, immunoliposomes, lipofectin, cationic facial amphiphiles (CFAs) and combinations thereof.

Physical injection of nucleic acid into cells represents the simplest gene delivery system (Vile & Hart (1994) Ann. Oncol, suppl. 5, 59). Accordingly, vectors comprising nucleotide sequences may be administered directly as “a naked nucleic acid construct” and may further comprise flanking sequences homologous to the host cell genome. After injection, nucleic acid is taken into cells and translocated to the nucleus, where it may be expressed transiently from an episomal location or stably if integration into the host genome occurs. Physical interventions may increase transfection efficiency, for example, focused ultrasound. The efficiency of transfection of cells in vivo may be increased by injecting DNA coated gold particles with a gene gun (Fyan et al. (1993) Proc. Natl. Acad. Sci. USA 90, 11478).

Liposomes are vesicles composed of phospholipid bilayer membranes that can enclose various substances, including nucleic acid. Mixtures of lipids and nucleic acid form complexes (lipoplexes) that can transfect cells in vitro and in vivo. Lipid mediated gene delivery has the ability to transfect various different cells without the need for interaction with specific receptors, minimal immunogenicity of the lipid components to facilitate multiple administration, high capacity vectors with the ability to deliver large DNA sequences and ease of production. The insertion of polyethylene glycol derivatives into the lipid membrane or pegylation may increase the circulation half-life of liposomes after administration. The pharmacokinetics, biodistribution and fusogenicity of liposomes may be varied by altering the composition of the lipid membrane. In particular, the incorporation of certain cationic lipids, for example, DMRIE, DOSPA and DOTAP with neutral or helper co-lipids—such as cholesterol or DOPE—in liposomes may increase their ability to fuse with cell membranes and deliver their contents into cells.

A number of nonlipid polycationic polymers form complexes with nucleic acid which promotes delivery into cells (Li and Huang (2000) Gene Ther. 7, 31). Preferably, the nonlipid polycationic polymers include but are not limited to poly-L-lysine, polyethylenimine, polyglucosamines and peptoids. Polyethylenimine may protect complexed nucleic acids from degradation within endosomes and it also provides a means of promoting nucleic acid release from the endosomal compartment and its subsequent translocation to the nucleus (Boussif et al. (1995) Proc. Natl. Acad. Sci. USA 92, 7297). Pegylated polyethylenimine polymers may decrease the interaction with serum proteins, extended circulation half-life and may deliver genes to cells without significant toxicity.

The transplantation of cells, for example, autologous, allogeneic and xenogeneic cells, that are genetically engineered to release biotherapeutic molecules may also be used. The transplanted cells may be surrounded with a permselective membrane that fully contains and protects them from attack by the host immune system. This method of encapsulation allows the neural transplantation of primary cells or cell lines from both allogeneic and xenogeneic sources. Various types of encapsulation techniques are known in the art. The method of microencapsulation allows the entrapment of small cell clusters within a thin, spherical, semipermeable membrane typically made of polyelectrolytes.

Viral Delivery

In a preferred embodiment, an IRES element is operably linked to one or more nucleotide sequences in a viral vector.

Viral delivery mechanisms are attractive vehicles for gene delivery since they have evolved specific and efficient means of entering human cells and expressing their genes.

Preferably, the viral genome is modified to remove sequences required for viral replication and pathogenicity. More preferably, the viral nucleotide sequences are replaced with one or more exogenous genes—such as an IRES element and one or more nucleotide sequences.

Viral delivery mechanisms include but are not limited to retrovirus, adenovirus, adeno-associated virus, herpes simplex virus, pox virus, lentiviral vectors, baculovirus, reovirus, Newcastle disease virus, alphaviruse and vesicular stomatitis virus vectors.

Retroviruses are single strand, diploid RNA viruses, which enter cells by binding surface envelope proteins, encoded by the env gene. After entering a cell, reverse transcriptase encoded by the pol gene transcribes the viral genome into a double strand DNA copy that can enter the nucleus of dividing cells and integrate randomly into the host genome. Preferably, retroviruses used for viral delivery are manipulated to render them replication deficient by removing their gag, pol and env genes. Thus, infectious but non-replicative retrovirus particles are produced in packaging cell lines that express retrovirus gag, pol and env genes from plasmids lacking a packaging sequence.

Insertion of IRES elements into retroviral vectors is compatible with the retroviral replication cycle and allows expression of multiple coding regions from a single promoter (Koo et al (1992) Virology 186:669-675; Chen et al 1993 J. Virol 67:2142-2148).

The lentiviruses, a subtype of retroviruses, may represent an alternative to retroviruses. Lentiviruses, such as HIV, simian and feline immunodeficiency viruses, can infect non-dividing cells and integrate in the same way as other retroviruses. Replication defective and multiply attenuated lentiviral vectors have been shown to lead to long term expression of various transgenes in the CNS of both rodents and primates (Bensadoun et al. (2000) Exp. Neurol. 164, 15-24; Kordower et al. (2000) Exp. Neurol. 160, 1-16). Lentiviral vectors diffuses 2-3mm from the injection site which allows the transduction of a significant number of neurones with a sustained gene expression up to at least one year.

Still other viruses are adenoviruses, which comprise double strand DNA viruses. More than 40 adenovirus serotypes in 6 groups (A to F) have been identified. Group C viruses (serotypes Ad2 and Ad5) have been most extensively evaluated as candidates for gene delivery (Zhang (1999) Cancer Gene Ther. 6, 11). Adenoviruses enter cells by binding to the coxsackievirus and adenovirus receptor, which facilitates interaction of viral arginine-glycine-aspartate (RGD) sequences with cellular integrins. After intemalisation, the virus escapes from cellular endosomes, partially disassembles and translocates to the nucleus, where viral gene expression begins. Preferably, the adenovirus is incapable of replication. This may be achieved by deleting one or more of the adenovirus genes—such as the early adenovirus genes E1 to E4. This may be extended to remove the whole nucleotide sequence of the adenovirus genome. Such viruses may be used for packaging a nucleotide sequence but must be grown in producer cell lines in the presence of helper viruses that supply all necessary viral gene functions to facilitate the packaging of infectious, replication incompetent adenovirus containing the nucleotide sequence.

Adeno-associated viruses are single strand DNA viruses that are native human viruses not known to cause any disease. They enter cells via binding to heparan sulfate but require co-infection with a so-called helper virus—such as adenovirus or herpes virus—to replicate. Adeno-associated virus vectors have a number of potential advantages. They infect non-dividing cells and are stably integrated and maintained in the host genome; integration occurs preferentially at a site dependent locus in chromosome 19, decreasing the risk of insertional mutagenesis. However, in adeno-associated virus vectors this characteristic integration is lost due to deletion of rep proteins in an attempt to decrease the risk of the emergence of replication competent adeno-associated viruses.

Herpes simplex viruses are large viruses with a linear double strand DNA genome of approximately 150 kbp that encodes more than 70 viral proteins. These viruses enter cells by binding viral glycoproteins to cell surface heparan sulfate residues. Preferably, herpes simplex viruses are rendered replication defective by inactivating a small number of genes—such as the immediate early genes ICPD, ICP4, 10P22 and ICP27. Since a large number of herpes simplex virus genes can be deleted without affecting the ability to produce viral vectors, large nucleic acid sequences containing multiple genes and their regulatory elements may be packaged within herpes simplex virus vectors.

Pox viruses are double strand DNA viruses that include vaccinia and canarypox or ALVAC. Preferably, the pox virus is a recombinant pox virus containing a nucleotide sequence.

Methods of delivery by viral techniques are now described in further detail below:

Adenovirus

One method for delivery involves the use of an adenovirus expression vector. Although adenovirus vectors are known to have a low capacity for integration into genomic DNA, this feature is counterbalanced by the high efficiency of gene transfer afforded by these vectors.

As used herein, the term “adenovirus expression vector” is meant to include those constructs containing adenovirus sequences sufficient to (a) support packaging of the construct and (b) to ultimately express a construct that has been cloned therein.

The vector comprises a genetically engineered form of adenovirus. Knowledge of the genetic organisation or adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan (1990) Radiother. Oncol. 19, 197-218). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

Recombinant adenovirus may be generated from homologous recombination between a shuttle vector and a provirus vector. Due to the possible recombination between two proviral vectors, wild-type adenovirus may be generated from this process. Therefore, it is critical to isolate a single clone of virus from an individual plaque and examine its genomic structure.

Generation and propagation of adenovirus vectors, which are replication deficient, depends on a helper cell line that constitutively expresses E1 proteins. Since the E3 region is dispensable from the adenovirus genome (Jones and Shenk (1978) Cell 131, 81-8.), adenovirus vectors with the aid of helper cells, may carry foreign DNA in either the E1, the D3 or both regions. In nature, adenovirus can package approximately 105% of the wild-type genome. providing capacity for about 2 extra kb of DNA. Combined with the approximately 5.5 kb of DNA that is replaceable in the E1 and E3 regions, the capacity of the current adenovirus vector is around 7.5 kb, or about 15% of the total length of the vector. More than 80% of the adenovirus viral genome remains in the vector backbone.

Helper cell lines may be derived from human cells—such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Various methods may be used for culturing helper cells and propagating adenovirus. In one format, natural cell aggregates are grown by inoculating individual cells into 1 litre siliconised spinner flasks (Techne, Cambridge, UK) containing 100-200 ml of medium. Following stirring at 40 rpm, the cell viability is estimated with trypan blue. In another format, Fibra-Cel microcarriers (Bibby Sterilin, Stone, UK) (5 g/l) are employed as follows. A cell inoculum, resuspended in 5 ml of medium, is added to the carrier (50 ml) in a 250 ml Erlemneyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus may be of any of the 42 different known serotypes or subgroups A-F. In some instances, the Adenovirus type 5 of subgroup C is the preferred starting material in order to obtain a conditional replication-defective adenovirus vector. This is because Adenovirus type 5 is a human adenovirus about which a great deal of biochemical and genetic information is known, and it has historically been used for most constructions employing adenovirus as a vector.

Thus, the typical vector is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors or in the E4 region where a helper cell line or helper virus complements the E4 defect.

Adenovirus growth and manipulation is known to those of skill in the art, and exhibits broad host range in vitro and in vivo. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Top et al. (1971) J Infect Dis. 124, 148-54), demonstrating their safety and therapeutic potential as in vivo gene transfer vectors.

Adenovirus vectors have been used in eukaryotic gene expression (Levrero et al. (1991) Gene 101, 195-202; Gomez-Foix et al. (1992) J Biol Chem. 267, 25129-34) and vaccine development (Graham & Prevec (1992)Biotechnol. 20. 363-90). Studies in administering recombinant adenovirus to different tissues include trachea instillation, muscle injection, peripheral intravenous injections and stereotactic inoculation into the brain (Le Gal La Salle et al. (1993) Science 12, 988-90).

Adeno-Associated Virus

Adeno-associated virus (AAV) may also be used in the present as it has a high frequency of integration and may even infect non-dividing cells. Details concerning the generation and use of AAV vectors are described in U.S. Pat. No. 5,139,941 and U.S. Pat. No. 4,797,368.

Recombinant AAV (rAAV) vectors have been used successfully for in vitro and in vivo transduction of marker genes (Kaplitt et al. (1994) Nat Genet 8, 148-54) and genes involved in human diseases.

AAV is a dependent parvovirus in that it requires coinfection with another virus (either adenovirus or a member of the herpes virus family) to undergo a productive infection in cultured cells. In the absence of coinfection with helper virus, the wild type AAV genome integrates through its ends into human chromosome 19 where it resides in a latent state as a provirus. rAAV, however, is not restricted to chromosome 19 for integration unless the AAV Rep protein is also expressed. When a cell carrying an AAV provirus is superinfected with a helper virus, the AAV genome is “rescued” from the chromosome or from a recombinant plasmid, and a normal productive infection is established (Muzyczka (1992) Curr. Top. Microbiol Immunol. 158, 97-129).

Typically, rAAV is made by cotransfecting a plasmid containing the gene of interest flanked by the two AAV terminal repeats and an expression plasmid containing the wild type AAV coding sequences without the terminal repeats. The cells are also infected or transfected with adenovirus or plasmids carrying the adenovirus genes required for AAV helper function. RAAV stocks made in such fashion are contaminated with adenovirus, which must be physically separated from the rAAV particles (for example, by caesium chloride density centrifugation). Alternatively, adenovirus vectors containing the AAV coding regions or cell lines containing the AAV coding regions and some or all of the adenovirus helper genes could be used.

AAV vectors have been successfully used for gene transfer into the brain of rodents and non-human primates (Peel & Kelin (2000) J Neurosci. Methods 98, 95-104). Owing to their low inflammation property they can be used to infect neurons in regions known to be very reactive such as the spinal cord.

Retrovirus

As mentioned above, the retroviruses are a group of single-stranded RNA viruses characterised by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from gag contains a signal for packaging of the genome into virions. Two long terminal repeat (LTR) sequences are present at the 5′ and 3′ ends of the viral genome. These contain strong promoter and enhancer sequences and are also required for integration in the host cell genome.

Pharmaceutical Compositions

Pharmaceutical compositions of the present invention may comprise a therapeutically effective amount of a vector.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the vector is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

The pharmaceutical composition comprising the vector of the present invention may also be used in combination with conventional treatments of diseases—such as conventional treatments for angiogenesis-dependent diseases.

The components may be administered alone but will generally be administered as a pharmaceutical composition—e.g. when the components are is in admixture with a suitable pharmaceutical excipient, diluent or carrier selected with regard to the intended route of administration and standard pharmaceutical practice.

For example, the components can be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, various sweetening or flavouring agents, colouring matter or dyes, with emulsifying and/or suspending agents and with diluents such as water, ethanol, propylene glycol and glycerin, and combinations thereof may be used.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Formulation

The component(s) may be formulated into a pharmaceutical composition, such as by mixing with one or more of a suitable carrier, diluent or excipient, by using techniques that are known in the art.

Host Cells

As used herein, the term “host cell” refers to any cell that comprises nucleotide sequences that are of use in the present invention.

Host cells may be transformed or transfected with a nucleotide sequence contained in a vector e.g. a cloning vector. The nucleotide sequence may be carried in a vector for the replication and/or expression of the nucleotide sequence. The cells will be chosen to be compatible with the said vector and may be eukaryotic cells (for example mammalian)—such as endothelial cells, or prokaryotic cells (for example bacterial), fungal, yeast or plant cells.

Transfection

Introduction of a vector into a host cell can be effected by various methods. For example, calcium phosphate transfection, DEAE-dextran mediated transfection, cationic lipid-mediated transfection, electroporation, transduction or infection may be used. Such methods are described in many standard laboratory manuals—such as Sambrook et al., Molecular Cloning: A Laboratory Manual, 2d ed. (1989) Cold Spring Harbor Laboratory Press, Cold Spring Harbor, N.Y.

Host cells containing the expression vector can be selected by using, for example, G418 for cells transfected with an expression vector carrying a neomycin resistance selectable marker.

Transformation

Teachings on the transformation of cells are well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

A host cell may be transformed with a nucleotide sequence. Host cells transformed with the nucleotide sequence may be cultured under conditions suitable for the replication or expression of the nucleotide sequence.

Variants/Homologues/Derivatives

As used herein, reference to SEQ ID No.1 and SEQ ID No. 2 also includes variants, homologues, derivatives and fragments thereof.

The term “variant” is used to mean a naturally occurring polypeptide or nucleotide sequences which differs from a wild-type sequence, but is functionally equivalent.

The term “fragment” indicates that a polypeptide or nucleotide sequence comprises a fraction of a wild-type sequence—such as the wild-type HC-IRES sequence. It may comprise one or more large contiguous sections of sequence or a plurality of small sections. The sequence may also comprise other elements of sequence, for example, it may be a fusion protein with another protein. Preferably, the sequence comprises at least 50%, more preferably at least 65%, more preferably at least 80%, most preferably at least 90% of the wild-type sequence.

The term “homologue” means an entity having a certain homology with the subject amino acid sequences and the subject nucleotide sequences. Here, the term “homology” can be equated with “identity”. Preferably, the homologue is functionally equivalent to the subject amino acid sequence.

In the present context, a homologous sequence is taken to include an amino acid sequence, which may be at least 75, 85 or 90% identical, preferably at least 95 or 98% identical to the subject sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence, which may be at least 75, 85 or 90% identical, preferably at least 95 or 98% identical to the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix—such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution—such as basic for basic, acidic for acidic, polar for polar etc. Non-homologous substitution may also occur i.e. from one class of residue to another or alternatively involving the inclusion of unnatural amino acids—such as omithine (hereinafter referred to as Z), diaminobutyric acid omithine (hereinafter referred to as B), norleucine omithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids—such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline , L-thioproline*, methyl derivatives of phenylalanine (Phe)—such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid# and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups—such as methyl, ethyl or propyl groups—in addition to amino acid spacers—such as glycine or β-alanine residues. A further form of variation involves the presence of one or more amino acid residues in peptoid form will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example, Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences may be modified by any method available in the art. Such modifications may be carried out to enhance the in vivo activity or life span of nucleotide sequences useful in the present invention.

The present invention may also involve the use of nucleotide sequences that are complementary to the nucleotide sequences or any derivative, fragment or derivative thereof. If the sequence is complementary to a fragment thereof then that sequence can be used as a probe to identify similar nucleotide sequences in other organisms.

Kits

The present invention also relates to kits for expressing one or more coding sequences in an endothelial cell.

In particular, the present invention relates to kits comprising one or more vectors according to the present invention for expressing one or more coding sequences in an endothelial cell.

In one embodiment, the kit comprises one or more vectors comprising one or more IRES elements operably linked to one or more coding sequences, wherein the IRES element expresses said coding sequences in an endothelial cell.

In another embodiment, the kit comprises one or more vectors comprising an endothelial cell ligand and one or more IRES elements operably linked to one or more coding sequences, wherein the IRES element expresses said coding sequences in an endothelial cell.

The present invention also provides kits that can be used in the methods of the present invention.

The kits of the present invention may include one or more control vectors. For example, the kit may comprise a positive control comprising an IRES element—such as the HC-IRES element—that is expressed in an endothelial cell. The kit may also comprise a negative control comprising an IRES element that is not expressed or is poorly expressed in an endothelial cell.

The kits of the present invention may also contain a means for detecting the expression of one or more coding sequences in an endothelial cell (eg. a detectable substrate such as a fluorescent compound, an enzymatic substrate, a radioactive compound or a luminescent compound).

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention will now be further described by way of Examples, which are meant to serve to assist one of ordinary skill in the art in carrying out the invention and are not intended in any way to limit the scope of the invention.

### EXAMPLES

### Example 1

Materials and Methods

Plasmid Preparation and Gene Targeting

The plasmids for homologous recombination in the Lmo2 gene were based on pKO5tk which has a unique BamHI restriction site mutated into exon 2 (12). The HC-IRES-lacZ Lmo2 knock-in targeting clone was prepared by inserting an HC-IRES-LacZ-MC1neopA cassette into the BamHI site of the pKO5tk. A 400 bp BamHI fragment including the IRES from the hepatitis C virus (18) (SEQ ID No.1 and SEQ ID No.2) was first cloned into the BglII-BamHI sites of a modified pBSpt vector (pBspt-BGB4) to generate the precursor pBSpt-HC-IRES with a unique BamHI site into which was cloned the lacZ gene and pMC1-neo-pA (19). The EMC-IRES Lmo2 knock-in targeting clone was prepared by inserting the lacZ gene fragment into pEMC IRES and addition of pMC1neo-pA and this cassette was cloned into pKO5tk. The in-frame fusion of lacZ with exon2 of the Lmo2 gene has been described previously and the generation and characterisation of the germ-line mouse carriers of the targeted allele (20).

Generation and Analysis of Gene Targeted Mice

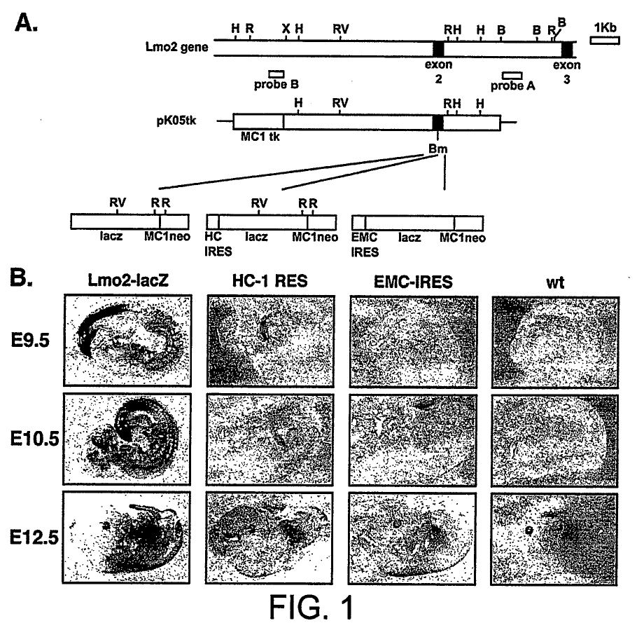

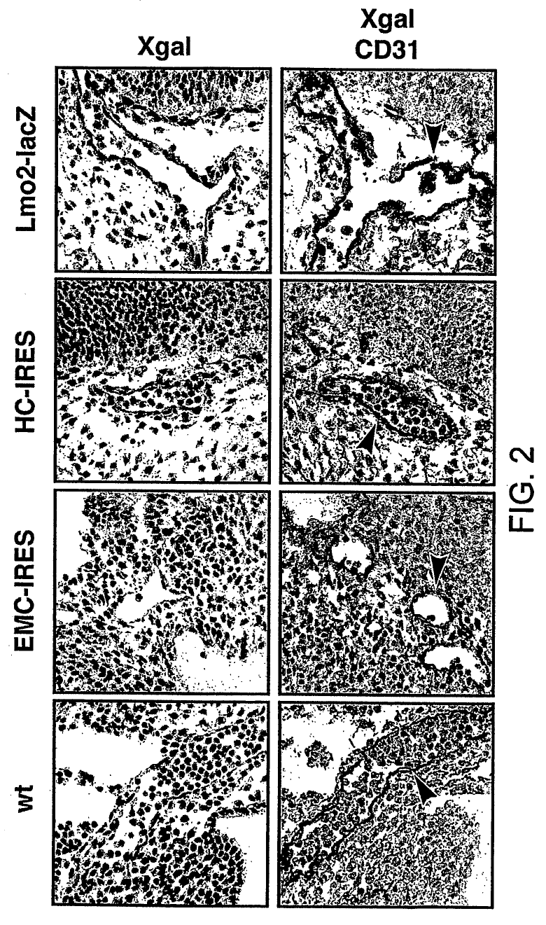

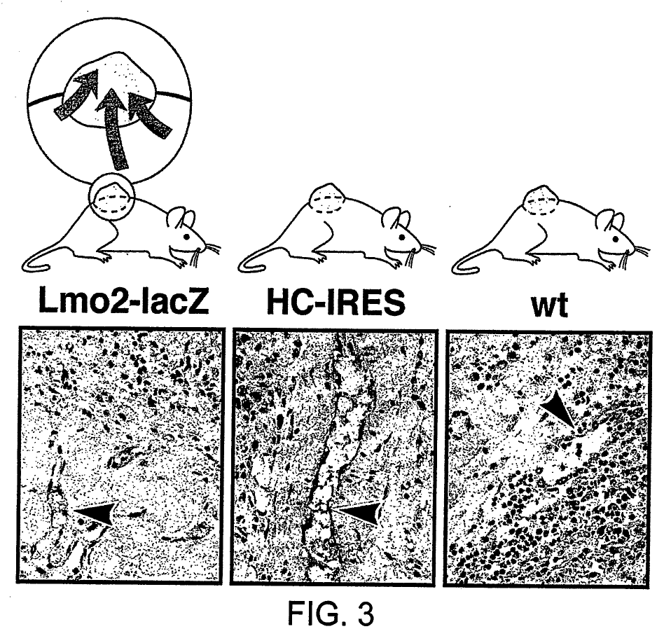

ES cells (CCB) were transfected and selected for G418 resistance and gancyclovir sensitivity as described (12) and targeted clones characterised by Southern filter hybridisation using two external probes, A and B (FIG. 1A). Targeted clones were injected into C57B16 blastocysts and chimaeric mice generated, from which germ-line transmission was obtained by breeding male chimaeras with C57B16 females. Timed matings were set up between heterozygous mice carrying one of the three Lmo2 knock-in alleles and wild type C57B16 mice. At the appropriate times, the pregnant females were euthanased, embryos removed and whole mount stained with X-gal to detect β-galactosidase as described (10). Post-fixed embryos (10% formalin) were sectioned after wax embedding. 4 μM sections were mounted on microscope slides and counter stained with haematoxylin and eosin. Detection of the endothelial marker PECAM (CD31) was carried out using MEC 13.3 anti-CD31 antibody (Pharmingen) by Avidin-Biotin conjugated peroxidase method as described (21).

Tumour Endothelial Cell Analysis

Lewis lung carcinoma cells were injected into both flanks of mice from each of the Lmo2 knock-in mouse lines or C57B16 controls (˜10⁶ cells per site). When primary site solid tumours reached about 1 cm size, the recipient mice were euthanased, tumours resected and whole mount X-gal staining carried out as for the embryos. After post-fixation in 10% formalin, sections were prepared from wax embedded specimens and 4 μM sections mounted, counter stained with haematoxylin and eosin.

### Example 2

Efficiency of HC-IRES in Vascular Endothelium during Embryogenesis

The ability of hepatitis C virus IRES element (HC-IRES) and EMC virus IRES to facilitate protein synthesis in blood vessel endothelial cells in during embryonic development was studied. The Lmo2 gene is expressed in and is necessary for sprouting endothelium in embryogenesis (9) and tumour growth (10). We chose this gene as a test situation for expressing bicistronic mRNA species in endothelial cells in vivo since the mouse Lmo2 gene is amenable to gene targeting in embryonic stem (ES) cells (12). We have created two lines of mice in which the expression of lacZ is controlled from an IRES element in the mRNA, namely the hepatitis C virus IRES or the encephalomyocarditis virus IRES (HC-IRES and EMC-IRES lines, respectively; FIG. 1A). In addition we compared the Lmo2-lacZ mouse line in which an in-frame fusion has been made between the lacZ gene and Lmo2 (9). Timed matings were established for the three lines and embryos were whole mount stained with Xgal to detect β-galactosidase activity at embryonic day E9.5, 10.5 and 12.5 (FIG. 1B). As previously reported (9), the developing vascular of the Lmo2-lacZ embryos expresses the Lmo2 gene which can readily be detected via the β-galactosidase reporter. No β-galactosidase activity was detected in wild type embryo litter mates (FIG. 1B). In the developing Lmo2-lacZ embryos, β-galactosidase is widely expressed in blood vessels being widely found in whole body developing vasculature which coincides with expression of the pan-endothelial marker PECAM/CD31, detected with anti-CD31 antibodies in histological sections of embryos at E10.5 (FIG. 2, top panels).

The levels of β-galactosidase reporter expression in the knock-in mouse lines with the Lmo2-HC-IRES-lacZ gene was less than the direct lacZ gene knock-into Lmo2 (FIG. 1B) but the detectable β-galactosidase in the blood vessel endothelial cells in the EMC-lacZ mice was very low and indeed virtually undetectable at embryonic day E10.5 (FIGS. 1B and 2). This suggests that the EMC virus IRES is unsuitable for endothelial expression in vivo. The hepatitis C viral IRES, on the other hand, yielded readily detectable levels of β-galactosidase activity. By the embryonic day E12.5, profound levels of endothelial expression had occurred indicating that the HC-IRES was used efficiently by the protein synthesis machinery of endothelial cells of mouse embryos.

### Example 3

The HC-IRES Mediates Endothelial Protein Synthesis in Tumour Angiogenesis

Angiogenesis is a target of cancer therapy (4,13,14), requiring targeting of anti-endothelial reagents to these specific cells. The efficacy of the HC-IRES in tumour blood vessels was tested using the lacZ knock-in mouse lines to support growth of tumour grafts which become vascularised by sprouting of existing blood vessels from the host. Lewis lung carcinoma cells were injected sub-cutaneously into the Lmo2-lacZ and HC-IRES mice (and C57B16 wild type controls) and solid tumours allowed to develop in situ at the site of injection. As the vascularisation of these tumours is contributed by the recipient mouse, the blood vessels endothelium would be expected to express the Lmo2-based lacZ reporter (FIG. 3A). This was analysed by staining isolated tumours with Xgal and histological sectioning to examine endothelial expression. FIG. 3B shows a comparison of sections made from Xgal stained tumours of the three sources showing that the Lmo2-lacZ and HC-IRES-transplanted tumours had comparable levels of β-galactosidase activity in this situation. The HC-IRES therefore has significant activity of in developing vasculature of tumours.

Discussion

There are number of important clinical indications where angiogenesis is an important consequence. Neovascularisation occurs around malignant tumours in order to supply enough oxygen and CO² exchange for rapidly dividing cells (14). In chronic inflammatory diseases such as rheumatoid arthritis, sustained inflammation results in the formation of vascular rich granulation tissues in the synovial membrane (1). Thus in these circumstances, preventing blood vessel remodelling and neovascularisation is a potential therapeutic approach (2,4,13). In circumstances where gene delivery is envisaged as a means of introducing proteins into target endothelial cells for therapy, the HC-IRES element could prove invaluable. In anti-angiogenesis therapies, a virus or other expression vector could encode therapeutics proteins (such as intracellular antibody fragments (15)) to two distinct intracellular targets, adding efficacy to the desired therapeutic effect. Alternatively, in solid tumour therapy, intracellular protein targets of angiogenesis, such as LMO2 (10), could be tackled by introduction of vectors encoding two blocking reagents aimed at prohibiting function of the target protein in distinct ways (for example, using an intracellular antibody fragment and a peptide aptamer (16)). Methods for delivery of vectors to specific cells in vivo are becoming more effective and specific ways of putting vectors into endothelial cells have been reported (17). Combining these delivery methods with the ability to efficiently express two or more proteins which can combat the function of specific targets is a possible approach to anti-angiogenesis therapies.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### REFERENCES

1. Carmeliet, P. and Jain, R. K. (2000) Angiogenesis in cancer and other diseases. Nature, 407, 249-257.
2. Folkman, J. (2001) Angiogenesis-dependent diseases. Semin Oncol, 28, 536-540.
3. Chang, Y. S., Di Tomaso, E., McDonald, D. M., Jones, R., Jain, R. and Munn, L. L. (2000) Mosaic blood vessels in tumors: frequency of cancer cells in contact with flowing blood. Proc. Natl. Acad, Sci. USA, 97, 14608-14613.
4. Kerbel, R. and Folkman, J. (2002) Clinical translation of angiogenesis inhibitors. Nat Rev Cancer, 2, 727-739.
5. Einfeld, D. A. and Roelvink, P. W. (2002) Advances towards targetable adenovirus vectors for gene therapy. Curr Opin Mol Ther, 4, 444-451.
6. Allen, T. M. (2002) Ligand-targeted therapeutics in anticancer therapy. Nat Rev Cancer, 2, 750-783.
7. Creancier, L., Morello, D., Mercier, P. and Prats, A. C. (2000) Fibroblast growth factor 2 internal ribosome entry site (IRES) activity ex vivo and in transgenic mice reveals a stringent tissue-specific regulation. J Cell Biol., 150, 275-281.
8. Creancier, L., Mercier, P., Prats, A. C. and Morello, D. (2001) c-myc Internal ribosome entry site activity is developmentally controlled and subjected to a strong translational repression in adult transgenic mice. Mol Cell Biol, 21, 1833-1840.
9. Yamada, Y., Pannell, R. and Rabbitts, T. H. (2000) The oncogenic LIM-only transcription factor Lmo2 regulates angiogenesis but not vasculogenesis. Proc. Natl. Acad. Sci. USA, 97, 320-324.
10. Yamada, Y., Pannell, R., Forster, A. and Rabbitts, T. H. (2002) The LIM-domain protein Lmo2 is a key regulator of tumour anogiogenesis: a new anti-angiogenesis drug target. Oncogene, 21, 1309-1315.
11. Garton, K. J., Ferri, N. and Raines, E. W. (2002) Efficient expression of exogenous genes in primary vascular cells using IRES-based retroviral vectors. Biotechniques, 32, 830-834.
12. Warren, A. J., Colledge, W. H., Carlton, M. B. L., Evans, M. J., Smith, A. J. H. and Rabbitts, T. H. (1994) The oncogenic cysteine-rich LIM domain protein rbtn2 is essential for erythroid development. Cell, 78, 45-58.
13. Folkman, J. (1971) Tumor angiogenesis: therapeutic implications. N. Eng. J. Med., 285,1182-1186.
14. Hanahan, D. and Folkman, J. (1996) Patterns and emerging mechanisms of the angiogenic switch during tumorigenesis. Cell, 86, 353-364.
15. Cattaneo, A. and Biocca, S. (1999) The selection of intracellular antibodies. Trends Biotechnol, 17(3), 115-21.
16. Colas, P., Cohen, B., Jessen, T., Grishina, I., McCoy, J. and Brent, R. (1996) Genetic selection of peptide aptamers that recognise and inhibit cyclin-dependent kinase 2. Nature, 380, 548-550.
17. Hood, J. D., Bednarski, M., Frausto, R., Guccione, S., Reisfeld, R. A., Xiang, R. and Cheresh, D. A. (2002) Tumor regression by targeted gene delivery to the neovasculature. Science, 296, 2404-2407.
18. Gallego, J. and Varani, G. (2002) The hepatitis C virus internal ribosome-entry site: a new target for antiviral research. Biochem Soc Trans, 30, 140-146.
19. Thomas, K. R. and Capecchi, M. R. (1987) Site-directed mutagenesis by gene targeting in mouse embryo-derived stem cells. Cell, 51, 503-512.
20. Yamada, Y., Pannell, R., Forster, A. and Rabbitts, T. H. (2000) The oncogenic LIM-only transcription factor Lmo2 regulates angiogenesis but not vasculogenesis. Proc. Natl. Acad. Sci. USA, 97, 320-324.
21. Taniere, P., Martel-Planche, G., Maurici, D., Lombard-Bohas, C., Scoazec, J.-Y., Montesano, R., Berger, F. and Hainaut, P. (2001) Molecular and Clinical Differences between Adenocarcinomas of the Esophagus and of the Gastric Cardia. Am. J. Pathol., 158, 33-40.

Sequences

SEQ ID No. 1

Nucleotide sequence of HC-IRES BamHI fragment
ggatccggcgacactccaccatgaatcactcccctgtgaggaactactgt
cttcacgcagaaagcgtctagccatggcgttagtatgagtgtcgtgcagc
ctccaggaccccccctcccgggagcgccatagtggtctgcggaaccggtg
agtacaccggaattgccaggacgaccgggtcctttcttgataaacccgct
caatgcctggagatttgggcgtgcccccgcaagactgctagccgagtagt
gttgggtcgcgaaaggccttgtggtactgcctgatagggtgcttgcgant
gccccgggaggtctcgtanaccgtgcaccatgagcacgaatctggatcc

SEQ ID No. 2

Amino acid sequence of HC-IRES BamHI fragment
GSGDTPPITPLGTTVFTQKASSHGVSMSVVQPPGPPLPGAPWSAEPVSTP
ELPGRPGPFLDKPAQCLEIWACPRKTASRVVLGRERPCGTAGACXCPGRS
RXPCTMSTNPGS

1. A vector comprising an endothelial cell ligand and one or more IRES elements operably linked to one or more coding sequences, wherein the IRES element expresses said one or more coding sequences in an endothelial cell.

2. The vector according to claim 1 wherein the IRES element comprises SEQ ID No. 1 or SEQ ID No. 2.

3. The vector according to claim 1 wherein the IRES element is a HC-IRES element.

4. The vector according to claim 1 wherein one or more IRES elements are operably linked to two or more coding sequences.

5. The vector according to claim 1 wherein the coding sequence(s) comprise therapeutic genes.

6. The vector according to claim I wherein the coding sequence(s) are expressed in endothelial cells in vitro or in vivo.

7. The vector according to claim 6 wherein the endothelial cells are diseased.

8. The vector according to claim 7 wherein the disease is an angiogenesis-dependent disease.

9. The vector according to claim 8 wherein the angiogenesis-dependent disease is characterized by excessive angiogenesis or insufficient angiogenesis.

10. The vector according to claim 1 wherein the endothelial cell ligand is a tumour endothelial cell ligand.

11. The vector according to claim 1 wherein the endothelial cell is a human endothelial cell.

12. The vector according to claim 1 wherein at least one of the coding sequences is under the control of an upstream promoter.

13. A method for expressing one or more coding sequences comprising the steps of:
(a) identifying an IRES element that expresses of one or more coding sequences in an endothelial cell;
(b) inserting the IRES element into a vector;
(c) transfecting the vector in to an endothelial cell; and
(d) providing for expression of the one or more coding sequences in the endothelial cell.

14. The method according to claim 13 wherein the IRES element comprises SEQ ID No. 1 or SEQ ID No. 2.

15. The method according to claim 13 wherein the IRES element comprises a HC-IRES element.

16. The method according to claim 13 wherein the one or more coding sequences are therapeutic genes.

17. The method according to claim 13 wherein the one or more coding sequences are expressed in endothelial cells in vitro or in vivo.

18. The method according to claims 13 wherein the endothelial cells are diseased.

19. The method according to claim 18 wherein the disease is an angiogenesis-dependent disease.

20. The method according to claim 19 wherein the angiogenesis-dependent disease is characterized by excessive angiogenesis or insufficient angiogenesis.

21. The method according to claim 13 wherein the endothelial cells are human endothelial cells.

22. The method according to claim 13 wherein the vector is a viral vector.

23. The method according to claim 13 wherein at least one of the coding sequences is under the control of an upstream promoter.

24. A method for preparing a vector for the expression of one or more coding sequences in an endothelial cell comprising the step of operably linking an IRES element to one or more coding sequences in a vector.

25. The method according to claim 24 comprising additional the steps of:
(a) transfecting the vector into an endothelial cell;
(b) providing for the expression of the one or more coding sequences; and
(c) determining whether the one or more coding sequences are expressed in the endothelial cell.

26. The method according to claim 24 wherein the IRES element comprises SEQ ID No. 1 or SEQ ID No. 2.

27. The method according to claim 24 wherein the IRES element comprises a HC-IRES element.

28. The method according to claim 24 wherein the one or more coding sequences are therapeutic genes.

29. The method according to claim 24 wherein the one or more coding sequences are expressed in endothelial cells in vitro or in vivo.

30. The method according to claims 24 wherein the endothelial cells are diseased.

31. The method according to claim 30 wherein the disease is an angiogenesis-dependent disease.

32. The method according to claim 31 wherein the angiogenesis-dependent disease is characterized by excessive angiogenesis or insufficient angiogenesis.

33. The method according to claim 24 wherein the endothelial cells are human endothelial cells.

34. The method according to claim 24 wherein the vector is a viral vector.

35. The method according to claim 24 wherein at least one of the coding sequences is under the control of an upstream promoter.

36. A method for identifying an IRES element that expresses one or more coding sequences in an endothelial cell comprising the steps of:
(a) operably linking an IRES element to one or more coding sequences in a vector;
(b) transfecting the vector into an endothelial cell;
(c) providing for expression of the one or more coding sequences; and
(d) determining whether the one or more coding sequences are expressed in the endothelial cell.

37. A method for delivering one or more coding sequences to an endothelial cell which comprises the step of transducing the endothelial cell with a vector according to claim 1.

38. A method for treating or preventing a disease in a subject, which comprises the step of administering a vector according to claim 1 to a subject.

39. A pharmaceutical composition comprising a therapeutically effective amount of a vector according to claim 1, and optionally a pharmaceutically acceptable carrier, diluent, excipient or adjuvant or any combination thereof.

40. A method according to claim 38, wherein the disease is an angiogenesis-dependent disease.

41. The method of claim 40 wherein the angiogenesis-dependent disease is characterized by excessive angiogenesis or insufficient angiogenesis.
